(19) United States
(12) Patent Application Publication
(10) Pub. No.: US 2016/0075667 A1
(43) Pub. Date: Mar. 17, 2016

(54) COMPOSITIONS AND METHODS FOR CHEMICAL EXCHANGE SATURATION TRANSFER (CEST) MAGNETIC RESONANCE IMAGING (MRI)

(71) Applicant: The Johns Hopkins University, Baltimore, MD (US)

(72) Inventors: Xing Yang, Baltimore, MD (US); Xiaolei Song, Baltimore, MD (US); Sangeeta Ray, Ellicott City, MD (US); Martin G. Pomper, Baltimore, MD (US); Michael T. McMahon, Columbia, MD (US)

(21) Appl. No.: 14/890,311

(22) PCT Filed: May 1, 2014
(86) PCT No.: PCT/US2014/036367

§ 371 Date: Nov. 10, 2015

Related U.S. Application Data
(60) Provisional application No. 61/822,001, filed on May 10, 2013.

(51) Int. Cl. C07D 249/04; C07D 231/14; A61K 49/10; C07D 233/90

(52) U.S. Cl. CPC C07D 249/04; C07D 233/90; C07D 231/14; A61K 49/10

(57) ABSTRACT

The invention features novel heterocyclic compounds that are useful as MRI contrast agents. Specifically, the invention relates to a novel class of MRI contrast agents that produce significantly improved contrast in MR images that is detectable through chemical exchange saturation transfer (CEST) or frequency labeled exchange (FLEX) imaging. The MRI contrast agents of the invention include those delineated in the formulae provided herein. The invention also relates to various methods in which the MRI contrast agents are employed. Kits and pharmaceutical compositions thereof are also provided.

### RELATED APPLICATIONS

This application claims the benefit of priority of U.S. Provisional Application No. 61/822,001, filed May 10, 2013, which is hereby expressly incorporated by reference in its entirety.

### STATEMENT REGARDING FEDERALLY-SPONSORED RESEARCH

The present invention was supported by grants from the National Institutes of Health (NIH), grant numbers EB 012590, EB 015031, and U54CA151838. The U.S. Government may have certain rights to the present invention.

### FIELD OF INVENTION

The present invention provides novel heterocyclic compounds that are useful as magnetic resonance imaging (MRI) contrast agents. The invention also provides a novel class of MRI contrast agents. The MRI contrast agents of the invention produce significantly improved contrast in magnetic resonance (MR) images in a pH dependent manner detectable through chemical exchange saturation transfer (CEST) or frequency labeled exchange (FLEX) imaging.

### BACKGROUND OF THE INVENTION

Chemical exchange saturation transfer (CEST) MR imaging is a technique in which low-concentration marker molecules are labeled by either saturating or labeling their exchangeable protons spins by radio-frequency (RF) irradiation. If such saturation or labeling can be achieved rapidly (i.e., before the spin exchanges), exchange of such labeled spins with water leads to transfer of the magnetization, allowing indirect detection of the solute via the water resonance through a change in signal intensity in MRI.

Each CEST contrast agent can have a different saturation frequency, which depends on the chemical shift of the exchangeable spin. The magnitude of proton transfer enhancement (PTE) due to this effect, and the resulting signal reduction from equilibrium (S₀) to saturated (S), are given by:

$$\begin{matrix}PTE = \frac{{NM}_{w} α {k}_{ex}}{( 1 - {x}_{CA} ) {R}_{1 wat } + {x}_{CA} {k}_{ex}} \cdot \{ 1 - {e}^{- [ ( 1 - {x}_{CA} ) {R}_{1 wat} + {x}_{CA} {k}_{ex} ] {t}_{sat}} \} , \\ and & [ Eq . 1 ] \\ ( 1 - {S}_{sat} / {S}_{0} ) = \frac{PTE \cdot [ CA ]}{2 \cdot [ {H}_{2} O ]} . & [ Eq . 2 ]\end{matrix}$$

“CA” is the contrast agent containing multiple exchangeable protons, x_{CA} its fractional exchangeable proton concentration, α the saturation efficiency, k the pseudo first-order rate constant, N the number of exchangeable protons per molecular weight unit, and M_{w} the molecular weight of the CA. The exponential term describes the effect of back exchange and water longitudinal relaxation (R_{1wat}=1/T_{1wat}) on the transfer during the RF saturation period (tₛₐₜ). This effect will be bigger when spins exchange faster, but the catch is that saturation must occur faster as well, which increases the radio-frequency power needed. In addition, the resonance of the particular spins must be well separated from the bulk in the NMR spectrum, which requires a slow exchange on the NMR time scale. This condition means that the frequency difference of the exchangeable spins with the bulk is much larger than the exchange rate (Δω>k).

Thus, the CEST technology becomes more applicable at higher magnetic fields or when using paramagnetic shift agents. Any molecule that exhibits a significant PTE effect can be classified as a CEST (contrast) agent. The concept of these agents as MR contrast agents is somewhat similar to the chemical amplification of colorimetric labels in in situ gene expression assays. For instance, CEST agents can be detected by monitoring the water intensity as a function of the saturation frequency, leading to a so-called z-spectrum. In such spectra, the saturation effect of the contrast agent on the water resonance is displayed as a function of irradiation frequency.

Since the first report of CEST contrast in 2000, CEST MR imaging has become widely used MRI contrast mechanism (demonstrated in FIG. 1). FIG. 1 shows that a CEST contrast is generated by the dynamic exchange process between an exchangeable proton of a biomarker of interest and the surrounding water protons. To detect the biomarkers, the magnetization of some of their exchangeable protons is nullified by applying a selective radiofrequency saturation pulse at the specific resonance frequency (chemical shift) of the target protons. Due to exchange of the “saturated” agent protons with surrounding water protons, the net water signal is reduced thus enhancing the MRI contrast.

CEST-MRI has been employed for many applications in molecular and cellular MRI. However, despite recent advances in the field of molecular magnetic resonance imaging (MRI), there is still a need for the design and development of MRI contrast agents that offer improved sensitivity and contrast effects in producing MR images.

### SUMMARY OF THE INVENTION

In one aspect, the present invention features novel heterocyclic compounds. In certain embodiments, the heterocyclic compounds of the invention are useful as magnetic resonance imaging (MRI) contrast agents.

In particular, the invention relates to a heterocyclic compound of formula (I), or a salt or stereoisomer thereof:

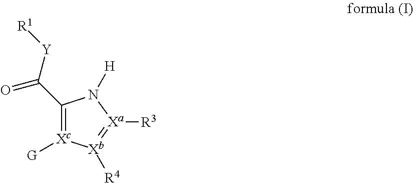

Wherein

X^{a}, X^{b}, and X^{c}, independently, are C, N, O, or S;

Y, on each occurrence, independently is alkyl, NR⁵, O, or S;

G is absent, H, alkyl, cycloalkyl, arylalkyl, cycloalkyl-alkyl, heterocyclic, heteroaryl-alkyl, aryl, heteroaryl, or

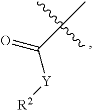

wherein said alkyl, cycloalkyl, arylalkyl, cycloalkyl-alkyl, heterocyclic, heteroaryl-alkyl, aryl, or heteroaryl moiety is optionally substituted;

R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;

R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or

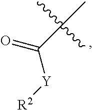

wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and

R⁵ is H, alkyl or —C(O)-alkyl;

provided that said compound is not one of the group of histidine; 4,5-imidazoledicarboxylic acid; 1H-tetrazole-5-acetic acid; and 4-imidazolecarboxylic acid.

In one embodiment, the heterocyclic compound of the invention is a compound of formula (II), or a salt or stereoisomer thereof;

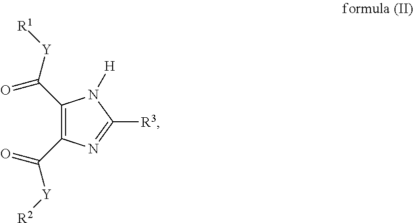

Wherein

Y, on each occurrence, independently is alkyl, NR⁵, O, or S;

R¹ and R², independently are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is H, halo, alkyl, cycloalkyl, arylalkyl, cycloalkyl-alkyl, heterocyclic, heteroaryl-alkyl, aryl, or heteroaryl, wherein said alkyl, cycloalkyl, arylalkyl, cycloalkyl-alkyl, heterocyclic, heteroaryl-alkyl, aryl, or heteroaryl moiety is optionally substituted; and

R⁵ is H or alkyl.

Certain exemplified compounds of the invention include, such as, compounds of the following group:

1) 4,5-bis[(Glu)carbonyl]-1H-imidazole (“I45DC-(Glu)₂”);

2) 4,5-bis[(Lys)carbonyl]-1H-imidazole (“I45DC-(Lys)₂”);

3) 4,5-bis[(Asp)carbonyl]-1H-imidazole (“I45DC-(Asp)₂”);

4) 3,5-bis[(Glu)carbonyl]-1H-pyrazole;

5) 4,5-bis[(Glu)carbonyl]-1H-1,2,3-triazole; and

6)

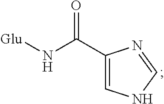

or a salt or stereoisomer thereof.

In another aspect, the invention relates to a novel class of MRI contrast agents. The MRI contrast agents of the invention include a compound of Formula (A), or a salt or stereoisomer thereof:

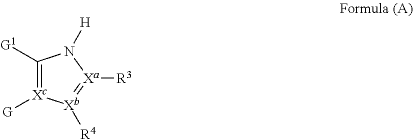

Wherein

X^{a}, X^{b}, and X^{c}, independently, are C, N, O, or S;

G¹ is H, alkyl, or

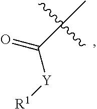

wherein the alkyl is optionally substituted;

G is absent, H, alkyl, cycloalkyl, arylalkyl, cycloalkyl-alkyl, heterocyclic, heteroaryl-alkyl, aryl, heteroaryl, or

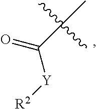

wherein said alkyl, cycloalkyl, arylalkyl, cycloalkyl-alkyl, heterocyclic, heteroaryl-alkyl, aryl, or heteroaryl moiety is optionally substituted;

Y, on each occurrence, independently is alkyl, NR⁵, O, or S;

R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;

R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or

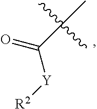

wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and

R⁵ is H, alkyl or —C(O)-alkyl.

Accordingly, one aspect of the invention provides a method of producing a magnetic resonance (MR) image of a target. The method comprises a step of introducing a MRI contrast agent of Formula (A) to the target.

Further, the invention features a method of diagnosing a tumor in a subject, wherein the method comprises the steps of a) introducing to the subject a MRI contrast agent of Formula (A) to obtain a conjugation of said MRI contrast agent and a tumor receptor; and b) detecting or sensing the conjugation. In one embodiment, the method further comprises the step of measuring a chemical shift change of exchangeable protons in the MRI contrast agent.

In another aspect of the invention, a method of detecting a pH value in a biological environment is provided. The method comprises the steps of a) introducing to said biological environment a MRI contrast agent of Formula (A); and b) measuring a chemical shift change of exchangeable protons in the MRI contrast agent.

In addition, the invention relates to a method of monitoring delivery of a pharmaceutically active agent in a subject. The method comprises the steps of a) administering to the subject the pharmaceutically active agent and a MRI contrast agent of Formula (A); and b) producing a magnetic resonance (MR) image of the pharmaceutically active agent.

The methods of the invention may further include a step of producing an image through chemical exchange saturation transfer (CEST)-based MRI technique. Alternatively, the image can be produced through frequency labeled exchange (FLEX) imaging technique.

In certain embodiments, the methods of the invention are pH dependent or pH sensitive.

Exemplified MRI contrast agents of Formula (A) are provided as follows:

1) 4,5-bis[(Glu)carbonyl]-1H-imidazole (“I45DC-(Glu)₂”);

2) 4,5-bis[(Lys)carbonyl]-1H-imidazole (“I45DC-(Lys)₂”);

3) 4,5-bis[(Asp)carbonyl]-1H-imidazole (“I45DC-(Asp)₂”);

4) 3,5-bis[(Glu)carbonyl]-1H-pyrazole;

5) 4,5-bis[(Glu)carbonyl]-1H-1,2,3-triazole;

6) 4,5-imidazoledicarboxylic acid;

7) 1H-tetrazole-5-acetic acid;

8) 4-imidazolecarboxylic acid;

9) imidazole;

10) 1H-1,2,3-triazole;

11) 1H-1,2,4-triazole;

12)

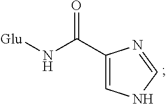

or a salt or stereoisomer thereof.

Also featured herein are kits that include one or more MRI contrast agents of Formulae (A), and instructions for producing an image thereof.

Still further, the invention features pharmaceutical compositions that contain an effective amount of a pharmaceutically active agent (e.g., a chemotherapeutic drug), and one or more MRI contrast agents of Formulae (A).

Also provided are MRI methods that embody the use of the MRI contrast agents of the invention.

Other aspects and embodiments of the invention are discussed below.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 demonstrates the contrast mechanism of CEST-MRI.

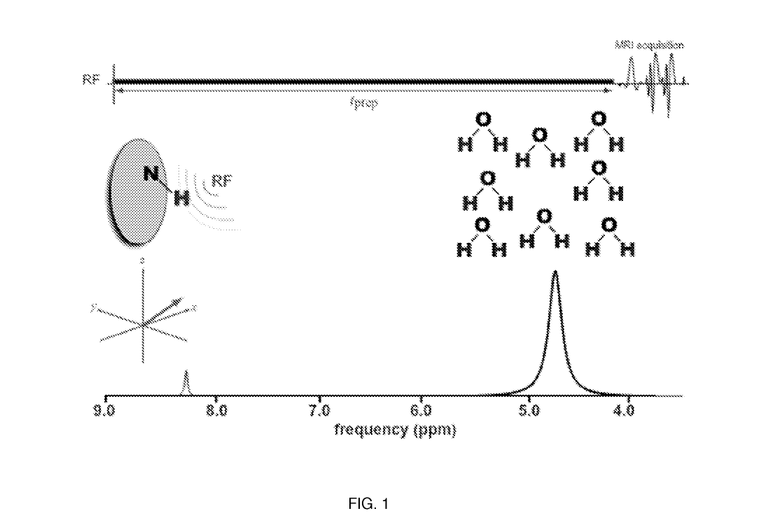

FIGS. 2A-2B depict CEST MTR_{asym} spectra for 100 mM I45DC-Glu at different pH values (A) and the exchange rate changes as a function of pH (B).

FIGS. 3A-3D depict (A) a Kidney anatomical image; (B) CEST maps at 7.5 ppm pre-i.v. injection of I45DC-Glu; (C) CEST maps at 7.5 ppm at 25 mins post-i.v. injection of I45DC-Glu; and (D) CEST maps at 7.5 ppm at 55 mins post-i.v. injection of I45DC-Glu.

FIG. 4 is a CEST MTR_{asym} spectrum of I45DC-(Glu)₂.

FIGS. 5A-5C provide (A) CEST MTR_{asym} spectra of I45DC-(Glu)₂; (B) a spectrum showing that the ratio of the contrast from I45DC-(Glu)₂ at 7.5 ppm to the contrast at 4.8 ppm is dependent on pH; and (C) pH mapping images of I45DC-(Glu)₂.

### DETAILED DESCRIPTION OF THE INVENTION

Compared with existing paraCEST contrast agents, organic CEST contrast agents offer potential advantages, such as, lower toxicity due to the absence of lanthanide metals, easy for modification, and clearance through breakdown during natural biochemical processes. However, currently reported organic CEST agents suffer from sensitivity drawbacks, probably due to a small chemical shift difference between exchangeable protons and water. For the best agents reported so far, the CEST protons resonate still below 6 ppm (e.g., Sherry et al., Annu. Rev. Biomed. Eng. 2008, 10, 391-411 etc).

The present inventors have discovered unexpectedly a novel class of organic compounds that are useful as MRI contrast agents. In particular, the present inventors have identified a novel class of heterocyclic compounds that are useful as MRI contrast agents. These heterocyclic compounds in general contain an imidazole-4,5-dicarboxamide (I45DC) scaffold, and thus are referred to as I45DCs in the present disclosure. The I45DCs of the invention can be used as diamagnetic CEST contrast agents, which offer the furthest reported shifted exchangeable protons (7.5 ppm) observed to date for organic small molecules.

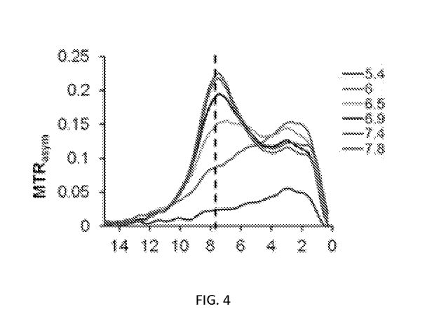

It was known that most azole N—H's have a relative high proton exchange rate (30,000 s⁻¹ or higher), which limits their practical application for existing CEST experimental protocols. To observe the CEST contrast, very high saturation power generally needs to be applied. The present inventors have designed a library of modified azole compounds and screened for their CEST contrast properties. The present inventors have found that a type of azole compounds, with the imidazole-4,5-dicarbonyl compounds (I45DCs), give a strong CEST contrast at 7.5 ppm from water while applying relatively low saturation power, with a proton exchangeable rate of ˜3500 s⁻¹. Further, the signal contrast showed a significant dependence on pH, which indicates that the compounds can be applied for tumor pH mapping (FIG. 4).

The present inventors have further discovered that the acid and base property of the compounds of the invention, especially the imidazoles, makes the compounds valuable pH sensors. Accordingly, certain embodiments of the invention provide that the MRI contrast agents of the invention produce significantly improved contrast in MR images in a pH dependent manner, which is detectable through CEST or FLEX imaging.

Further, the present inventors discovered that the CEST contrast produced from the imidazole-4,5-dicarboxamide scaffolds was tolerant to different chemical modifications.

The MRI contrast agents of the invention can be used for various clinical or non-clinical purposes, including, but not limited to, determining intratumoral pH, determining encapsulated cell pH, determining kidney pH, monitoring the delivery of chemotherapeutics, or for targeted imaging studies through conjugation of a receptor ligand or an antigen.

### DEFINITIONS

Before further description of the invention, and in order that the invention may be more readily understood, certain terms are first defined and collected here for convenience.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “administration” or “administering” includes routes of introducing the compound of the invention to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The phrase “in combination with” is intended to refer to all forms of administration that provide a compound of the invention (e.g. a compound selected from any of the formulae described herein) together with a second agent, such as a second compound selected from any of the formulae described herein, or an existing therapeutic agent used for a particular disease or disorder, where the two are administered concurrently or sequentially in any order.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes divalent alkyl (e.g., —CH₂— etc) groups and can further include oxygen, nitrogen, sulfur or phosphorous atoms replacing one or more carbons of the hydrocarbon backbone, e.g., oxygen, nitrogen, sulfur or phosphorous atoms. For convenience, C₀alkyl used herein refers to a bond or a H atom.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

The term “alkoxy” refer to a —O-alkyl group.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, with said heteroatoms selected from O, N, and S, and the remainder ring atoms being carbon. Heteroaryl groups may be optionally substituted with one or more substituents. Examples of heteroaryl groups include, but are not limited to, pyridyl, furanyl, benzodioxolyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, and indolyl. In one embodiment of the invention, heteroaryl refers to thienyl, furyl, pyridyl, or indolyl.

The term “associating with” refers to a condition of proximity between a chemical entity or compound, or portions thereof, and a binding pocket or binding site on a protein. The association may be non-covalent (wherein the juxtaposition is energetically favored by hydrogen bonding or van der Waals or electrostatic interactions) or it may be covalent.

The language “biological activities” of a compound of the invention includes all activities elicited by compound of the invention in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

By “agent” is meant a polypeptide, polynucleotide, cell, or fragment, or analog thereof, small molecule, or other biologically active molecule.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “heterocyclic group” includes closed ring structures in which one or more of the atoms in the ring is an element other than carbon, for example, nitrogen, sulfur, or oxygen. Heterocyclic groups can be saturated or unsaturated and heterocyclic groups, such as, pyrrole and furan can have aromatic character. They include fused ring structures, such as, quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like. Suitable heteroaromatic and heteroalicyclic groups generally will have 1 to 3 separate or fused rings with 3 to about 8 members per ring and one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino, and pyrrolidinyl.

The language “improved properties” refers to any activity associated with a MRI contrast agent of the invention that reduces its toxicity and/or enhances its effectiveness or sensitivity for producing MR images in vitro or in vivo. In one embodiment, this term refers to any qualitative or quantitative improved property of a compound of the invention, such as, reduced toxicity.

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈ alkenyl, C₂-C₈alkynyl, C₁-C₈alkoxy, C₂-C₈alkyl ether, C₃-C₈alkanone, C₁-C₈alkylthio, amino, mono- or di-(C1-C₈alkyl)amino, haloC₁-C₈alkyl, haloC₁-C₈alkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “isomers” or “stereoisomers” refers to compounds which have identical chemical constitution, but differ with regard to the arrangement of the atoms or groups in space.

The term “obtaining” as in “obtaining a compound” is intended to include purchasing, synthesizing or otherwise acquiring the compound.

The term “subject” includes organisms which are capable of suffering from any disease or disorder, which could be detected or sensed from the administration of a MRI contrast agent of the invention. It is also contemplated that the subject may be an artificial system which mimics biological environment of a living organism. The “subject” includes a living organism, such as, human, non-human animals, fungus, micro-organism, or plant. Preferred humans include human patients as identified in need thereof. The term “non-human animals” of the invention includes all vertebrates, e.g., mammals, e.g., monkeys, rodents, mice, and non-mammals, such as non-human primates, e.g., sheep, dog, cow, chickens, amphibians, reptiles, etc.

### Compounds of the Invention

The invention provides novel heterocyclic compounds that can be potentially used as MRI contrast agent(s).

In certain embodiments, the invention features a compound of Formula (I), or a salt or stereoisomer thereof:

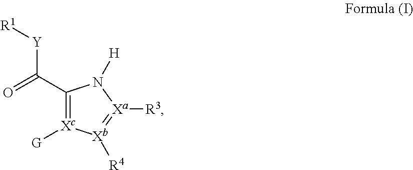

### Wherein

X^{a}, X^{b}, and X^{c}, independently, are C, N, O, or S;

Y, on each occurrence, independently is alkyl, NR⁵, O, or S;

G is absent, H, alkyl, cycloalkyl, arylalkyl, cycloalkyl-alkyl, heterocyclic, heteroaryl-alkyl, aryl, heteroaryl, or

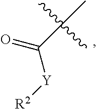

wherein said alkyl, cycloalkyl, arylalkyl, cycloalkyl-alkyl, heterocyclic, heteroaryl-alkyl, aryl, or heteroaryl moiety is optionally substituted;

R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;

R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or

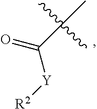

wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and

R⁵ is H, alkyl or —C(O)-alkyl;

provided that said compound is not one of the group of histidine; 4,5-imidazoledicarboxylic acid; 1H-tetrazole-5-acetic acid; and 4-imidazolecarboxylic acid.

A specific embodiment of formula (I) provides that G is

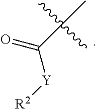

In certain embodiments, X^{c} is C.

In one embodiment, X^{a} is C. In another embodiment, X^{a} is N.

Certain embodiments of Formula (I) feature a compound of formula (II) or a salt or stereoisomer thereof:

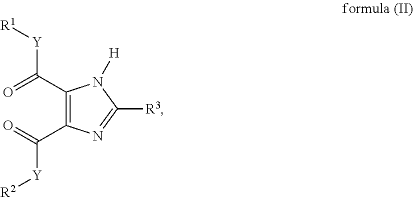

### Wherein

Y, on each occurrence, independently is alkyl, NR⁵, O, or S;

R¹ and R², independently are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is H, halo, alkyl, cycloalkyl, arylalkyl, cycloalkyl-alkyl, heterocyclic, heteroaryl-alkyl, aryl, or heteroaryl, wherein said alkyl, cycloalkyl, arylalkyl, cycloalkyl-alkyl, heterocyclic, heteroaryl-alkyl, aryl, or heteroaryl moiety is optionally substituted; and

R⁵ is H or alkyl.

One embodiment provides that R³ is H. In certain embodiments, both Ys are NH.

In a separate embodiment, R¹ and R², independently, are (C₁₋₃)alkyl that is optionally substituted by one or more substituents selected from the group of a carboxylic group, an ester group, an amino group, and an amide group. For example, R¹ and R², each independently, can be one of the following:

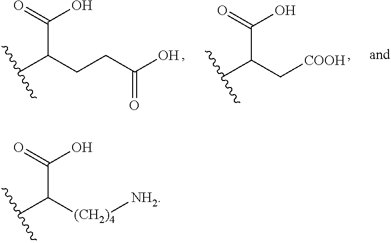

It is featured in one embodiment that R¹ and R² are the same. Alternatively, R¹ and R² are different from each other.

Exemplified compounds of the invention include, but are not limited to, compounds as follows:

1)

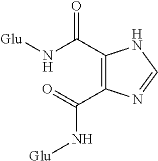

4,5-bis[(Glu)carbonyl]-1H-imidazole (also referred to as “I45DC-(Glu)2”);

2)

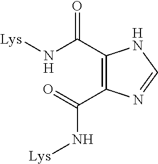

4,5-bis[(Lys)carbonyl]-1H-imidazole (also referred to as “I45DC-(Lys)2”); and

3)

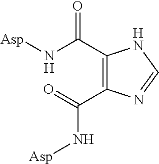

4,5-bis[(Asp)carbonyl]-1H-imidazole (also referred to as “I45DC-(Asp)2”);

or a salt or stereoisomer thereof.

Certain embodiments of formula (I) provide that X^{c} is C, and X^{a} is N.

One embodiment provides that G is

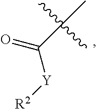

and R⁴ is H or absent. Another embodiment features that G is H, and R⁴ is

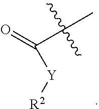

Further embodiments provide that Y is NH at each occurrence.

When applicable, R¹ and R² can be the same or different. One embodiment provides that R¹ and R², each independently, are (C₁₋₃)alkyl that is optionally substituted by one or more substituents selected from the group of a carboxylic group, an ester group, an amino group, and an amide group. For example, R¹ and R², each independently, can be one of the following:

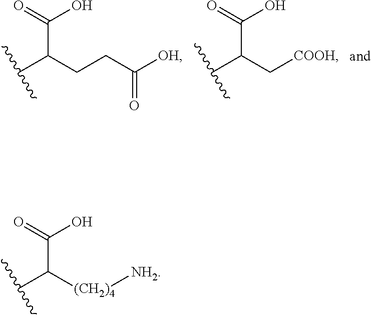

The invention further features the following exemplified compounds:

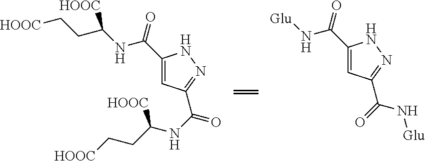

3,5-bis[(Glu)carbonyl]-1H-pyrazole; and

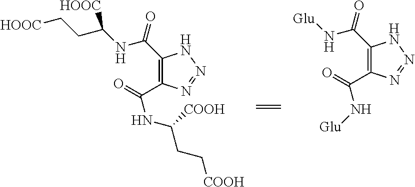

4,5-bis[(Glu)carbonyl]-1H-1,2,3-triazole;

or a salt or stereoisomer thereof.

A further embodiment of formula (I) provides that G is H. One embodiment features that R³ is H, and X^{c} is C. Still another embodiment provides that X^{b} is N, and R⁴ is absent.

An exemplified compound herein is

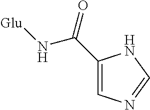

(S)-2-(1H-imidazole-5-carboxamido)pentanedioic acid,
   or a salt or stereoisomer thereof.

Further, the invention also relates to a compound of compound of Formula (A), or a salt or stereoisomer thereof:

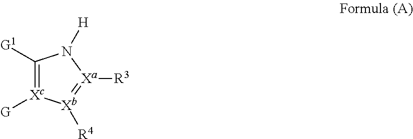

### Wherein

X^{a}, X^{b}, and X^{c}, independently, are C, N, O, or S;

G¹ is H, alkyl, or

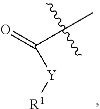

wherein the alkyl is optionally substituted;

G is absent, H, alkyl, cycloalkyl, arylalkyl, cycloalkyl-alkyl, heterocyclic, heteroaryl-alkyl, aryl, heteroaryl, or

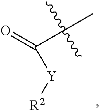

wherein said alkyl, cycloalkyl, arylalkyl, cycloalkyl-alkyl, heterocyclic, heteroaryl-alkyl, aryl, or heteroaryl moiety is optionally substituted;

Y, on each occurrence, independently is alkyl, NR⁵, O, or S;

R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;

R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;

R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or

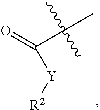

wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and

R⁵ is H, alkyl or —C(O)-alkyl.

In certain embodiments, at least one of X^{a}, X^{b}, and X^{c} is N. In another embodiment, R³ is H.

One embodiment provides that G¹ is H. Another embodiment provides that G¹ is (C₁₋₆)alkyl that is optionally substituted by one or more substituents, such as, a hydroxyl group, a carboxylic group, and an amino group.

In still another embodiment, G¹ is

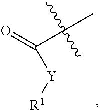

wherein Y is N or O, and R¹ is H or (C₁₋₆)alkyl optionally substituted by one or more substituents, such as, a hydroxyl group, a carboxylic group, and an amino group.

The invention also features a stereoisomer (e.g., a regio-isomer, diastereomer, and enantiomer etc.), a salt, ester, hydrate, precursor, derivative, polymorph, or solvate thereof of a compound of the above formulae.

For example, suitable salts that can be used include those well known in the art (see, e.g., Berge et al. (1977) “Pharmaceutical Salts”, J. Pharm. Sci. 66:1-19). Such a salt can be an inorganic salt or an organic salt. The inorganic salt can be, e.g., a metal salt including, but not limited to, a sodium salt, a potassium salt, and a cesium salt, and etc.

Also, the compounds of the invention may contain one or more asymmetric centers and thus occur as racemates and racemic mixtures, single enantiomers, individual diastereomers and diastereomeric mixtures. All such isomeric forms of these compounds are expressly contemplated. The compounds of the invention may also be represented in multiple tautomeric forms, in such instances, the invention expressly includes all tautomeric forms of the compounds described herein. All such isomeric forms of such compounds are expressly included. Crystal forms of the compounds described herein are also included.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The compounds of the invention can be prepared according to a variety of methods, some of which are known in the art. Methods of synthesizing the compounds of the invention are exemplified in Example 1; other methods of preparation will be apparent to one of ordinary skill in the art. Methods for optimizing reaction conditions, if necessary minimizing competing by-products, are known in the art. The methods may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

In general, the compounds of the invention possess CEST contrast properties. Thus, the compounds of the invention are useful as MRI contrast agents. In particular, the present inventors have identified that the CEST contrast properties from the imidazole-4,5-dicarboxamide scaffolds were tolerant to different chemical modifications. Thus, a variety of I45DCs have been synthesized and/or obtained.

It has been discovered that I45DCs of the invention possess various properties, including pH sensitivity of the contrast, cell penetration capabilities, in vivo pharmacokinetics, binding to specific targets in vivo, etc. These I45DC compounds can be used for various purposes including but not limited to determining intratumoral pH, determining encapsulated cell pH, determining kidney pH, monitoring the delivery of chemotherapeutics, or for targeted imaging studies through conjugation of a receptor ligand or an antigen.

### Methods and Kits

In one aspect, the invention relates to a method of producing a magnetic resonance (MR) image of a target. The method comprises a step of introducing a MRI contrast agent of the invention to the target.

Certain embodiments of the invention provide that the target is a tumor, a biological tissue, a ligand, a therapeutically active agent, or a metal ion.

Further, the invention features a method of diagnosing a tumor in a subject, wherein the method comprises the steps of a) introducing to the subject a MRI contrast agent of the invention to obtain a conjugation of said MRI contrast agent and a tumor receptor; and b) detecting or sensing the conjugation. In one embodiment, the method further comprises the step of measuring a chemical shift change of exchangeable protons in the MRI contrast agent.

In another aspect of the invention, a method of detecting a pH value in a biological environment is provided. The method comprises the steps of a) introducing to said biological environment a MRI contrast agent of the invention; and b) measuring a chemical shift change of exchangeable protons in the MRI contrast agent.

In addition, the invention relates to a method of monitoring delivery of a pharmaceutically active agent in a subject. The method comprises the steps of a) administering to the subject the pharmaceutically active agent and a MRI contrast agent of the invention; and b) producing a magnetic resonance (MR) image of the pharmaceutically active agent.

One embodiment provides that the pharmaceutically active agent and the MRI contrast agent are administered concurrently to the subject. Alternatively, the pharmaceutically active agent and the MRI contrast agent are administered sequentially; i.e., the MRI contrast agent is administered before or after the administration of the pharmaceutically active agent. It is also contemplated that the pharmaceutically active agent and the MRI contrast agent can be pre-mixed before the administration.

According to certain embodiments of the invention, the subject is a subject identified as in need thereof by one of ordinary skills in the art. In certain embodiments, the subject includes human and non-human animals. In one embodiment, the subject is a human. In another embodiment, the subject is a non-human mammal (e.g., monkeys, rodents, and mice). A separate embodiment provides that the subject is an artificial system which mimics biological environment of a living organism.

The methods of the invention may further include a step of producing an image through chemical exchange saturation transfer (CEST)-based MRI technique. Other embodiments provide that the image is produced through frequency labeled exchange (FLEX) imaging technique.

According to the invention, the MRI contrast agent used here is a heterocyclic compound, especially, a compound of any one of the above-noted formulae. In a certain embodiment, the MRI contrast agent is a compound of formula (A). In another embodiment, the MRI contrast agent is a compound of formula (I) or (II).

In certain embodiments, the MRI contrast agents of the invention are pH sensitive or pH dependent. Thus, certain embodiments provide that the methods of the invention are pH sensitive or pH dependent.

Specific MRI contrast agents of the invention include, but are not limited to, compounds of the following:

1)

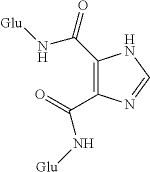

4,5-bis[(Glu)carbonyl]-1H-imidazole (“I45DC-(Glu)₂”);

2)

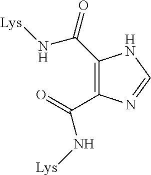

4,5-bis[(Lys)carbonyl]-1H-imidazole (“I45DC-(Lys)₂”);

3)

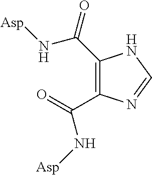

4,5-bis[(Asp)carbonyl]-1H-imidazole (“I45DC-(Asp)₂”);

4)

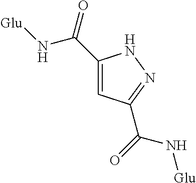

3,5-bis[(Glu)carbonyl]-1H-pyrazole;

5)

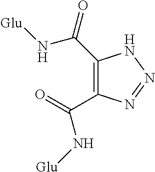

4,5-bis[(Glu)carbonyl]-1H-1,2,3-triazole;

6)

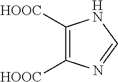 [SMILES: O=C(O)c1nc[nH]c1C(=O)O]

4,5-imidazoledicarboxylic acid;

7)

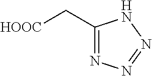

1H-tetrazole-5-acetic acid;

8)

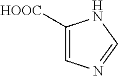

4-imidazolecarboxylic acid;

9)

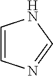

imidazole;

10)

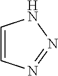

1H-1,2,3-triazole;

11)

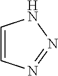

1H-1,2,4-triazole;

12)

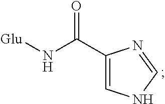

or a salt or stereoisomer thereof.

In a specific embodiment, the MRI contrast agent of the invention is one of the following

1) 4,5-bis[(Glu)carbonyl]-1H-imidazole (“I45DC-(Glu)₂”);

2) 4,5-bis[(Lys)carbonyl]-1H-imidazole (“I45DC-(Lys)₂”); and

3) 4,5-bis[(Asp)carbonyl]-1H-imidazole (“I45DC-(Asp)₂”);

or a salt or stereoisomer thereof.

The MRI contrast agents of the invention also include a stereoisomer (e.g., a regio-isomer, diastereomer, and enantiomer etc.), salt, ester, hydrate, precursor, derivative, polymorph, or solvate thereof of a compound as above delineated.

According to certain embodiments of the invention, significantly improved contrast in MR images can be produced. In certain embodiments, the contrast in MR images is produced in a pH dependent manner detectable through chemical exchange saturation transfer (CEST) or frequency labeled exchange (FLEX) imaging. The MRI contrast agents of the invention can be used for various purposes including but not limited to determining intratumoral pH, determining encapsulated cell pH, determining kidney pH, or for targeted imaging studies through conjugation of a receptor ligand or antigen.

The methods of the invention are useful for detecting, sensing, or imaging various types of material (e.g., enzymes, vitamins, ligands, tissues, metal ions, organic substrates, and biologically active chemical elements).

Also provided is a method of identifying a compound useful as a MRI contrast agent, which includes a step of screening the compound for its CEST properties. In certain embodiments, the compound is a heterocyclic compound, e.g., an azole compound.

The invention also includes a method of designing and/or preparing (e.g., synthesizing) compounds that are useful as MRI contrast agents. The method comprises one or more following steps: evaluating the structures of existing MRI contrast agents for their CEST contrast properties, designing and synthesizing new compounds, and screening the new compounds for their CEST contrast properties.

The CEST approach of the invention can be further extended to designing of other novel responsive agents for molecular and cellular MRI applications. Any potential novel responsive agents may be assessed by an optical assay (using multi-well plates) for their potentiality for the CEST approach.

Certain design criteria for creating MRI contrast agents can be found in Que et al. (Chem Soc. Rev. 2010, 39, 51-60) and Hyman et al. (Coordination Chemistry Reviews, 256 (2012), 2333-2356).

Further featured are methods that embody the use of the MRI contrast agents of the invention.

The invention also provides a kit that includes one or more MRI contrast agents of the invention, and instructions for producing an image thereof.

Still further, the invention features pharmaceutical compositions that contain an effective amount of a pharmaceutically active agent (e.g., a chemotherapeutic drug), and one or more MRI contrast agents of the invention.

### EXAMPLES

### General Reagents and Analyses

All chemicals and solvents were purchased from Sigma-Aldrich (Milwaukee, Wis.). The imidazole, L-histidine, 1H-1,2,3-trazole, 1H-1,2,4-triazole, 1H-tetrazole-5-acetic acid, 4-imidazolecarboxylic acid and 4,5-imidazoledicarboxylic acid used in screening tests were purchased from Sigma-Aldrich (Milwaukee, Wis.).

¹H NMR and ¹³C NMR spectra were obtained on a Bruker Avance 400 MHz Spectrometer (Billerica, Mass.). Mass spectra were obtained on a Bruker Esquire 3000 plus system (ESI) or an Applied Biosystems Voyager DE-FTR MALDI-TOF (Foster City, Calif.). High-performance liquid chromatography (HPLC) purifications were performed on an Agilent 1260 Infinity preparative HPLC system from Agilent (Santa Clara, Calif.).

### Example 1

### Synthesis, Characterization Data and HPLC Properties of Azole CEST Contrast Agents

In general, symmetrical or unsymmetrical I45DCs could be synthesized by reacting free amines with 5,10-dioxo-5H,10H-diimidazo[1,5-a:1′-5′-d]-pyrazine-1,6-dicarboxylic acid diphenyl ester, according to the following scheme:

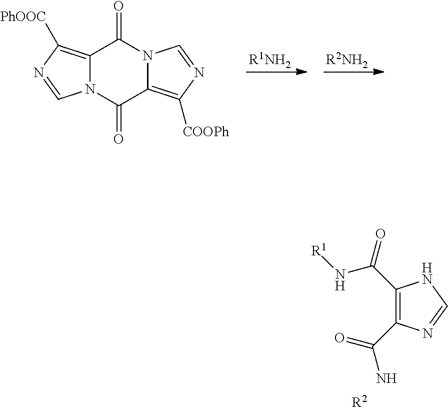

### a) Synthesis of (S)-2-(1H-imidazole-5-carboxamido)pentanedioic acid

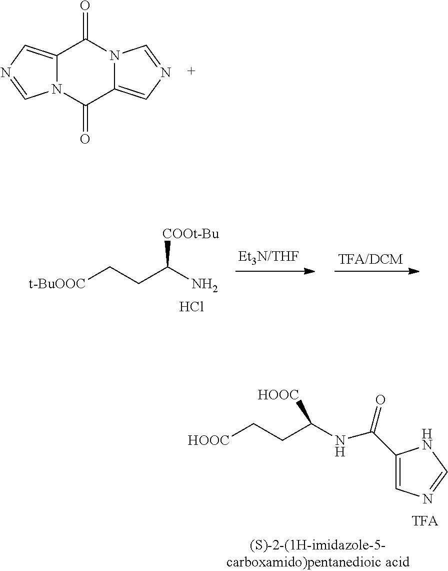

Diimidazo[1,5-a]piperazine-5,10-dione 376 mg (2 mmol) was dissolved in 20 mL dry THF. H-Glu(Ot-Bu)Ot-Bu HCl 1.2 g (4 mmol) and triethyl amine 3 mL (20 mmol) were added to the solution at 0° C. and the reaction was stirred overnight at room temperature. After the solvent was removed under vacuum, the tert-butyl protected intermediate was obtained by flash column chromatography. Then, this intermediate was dissolved in 5 mL TFA/DCM (1/1) for 2 hours at room temperature. After all the solvent was removed under vacuum, compound (S)-2-(1H-imidazole-5-carboxamido)pentanedioic acid was purified by HPLC as a white powder 310 mg, yield 23%. ¹H NMR (400 MHz, D₂O): δ 8.69 (s, 1H), 7.91 (s, 1H), 4.47 (dd, J1=7.2 Hz, J2=3.9 Hz, 1H), 2.38 (t, J=5.4 Hz, 2H), 2.20-2.11 (m, 1H), 2.01-1.94 (m, 1H) ¹³C NMR (100 MHz, D₂O): δ177.0, 174.6, 162.8 (q, TFA), 158.7, 135.5, 126.6, 120.7, 116.4 (q, TFA), 52.4, 29.8, 25.6; HPLC (Waters Atlantis, MeCN/H₂O 8/92, 10 mL/min): 9.5 min.

### b) Synthesis of 4,5-bis[(Glu)carbonyl]-1H-imidazole (I45DC-(Glu)₂), 4,5-bis[(Lys)carbonyl]-1H-imidazole (I45DC-(Lys)₂) and 4,5-bis[(Asp)carbonyl]-1H-imidazole (I45DC-(Asp)₂)

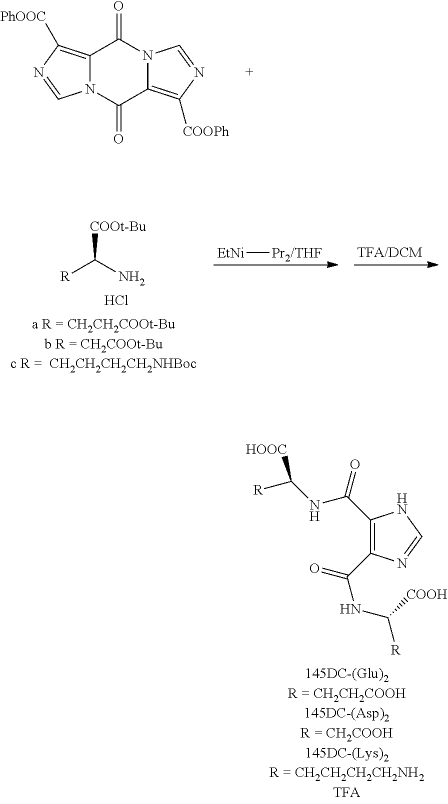

5,10-Dioxo-5H,10H-diimidazo[1,5-a:1′-5′-d]pyrazine-1,6-dicarboxylic Acid Diphenyl Ester, 214 mg (0.5 mmol) and 5 mL THF were added to a dry flask. To this suspension at 0° C. was added the protected amino acids (a-c) 1 mmol and EtNi-Pr₂ 2 mL (11 mmol). After stirring at room temperature for 2 hours, the reaction was refluxed for 2 to 4 days, monitored by TLC. After the solvent was removed under vacuum, the tert-butyl protected intermediate was obtained by flash column chromatography. Then, this intermediate was dissolved in 5 mL TFA/DCM (1/1) for 2 hours at room temperature. After all the solvent was removed under vacuum, compound I45DCs was purified by HPLC.

I45DC-(Glu)₂ with a yield of 66% as a white powder: ¹H NMR (400 MHz, D₂O): δ 7.89 (s, 1H), 4.57 (dd, J1=6.6 Hz, J2=3.6 Hz, 2H), 2.45 (t, J=5.4 Hz, 4H), 2.25-2.22 (m, 2H), 2.09-2.05 (m, 2H); ¹³C NMR (100 MHz, D₂O): δ 176.9, 174.5, 161.4, 136.9, 129.8, 52.1, 29.9, 26.0; HPLC (Waters Atlantis, MeCN/H₂O 15/85, 6 mL/min): 15.0 min.

I45DC-(Asp)₂ with a yield of 58% as a white powder: ¹H NMR (400 MHz, D₂O): δ 7.84 (s, 1H), 4.87 (t, J=3.9 Hz, 2H), 3.05-2.91 (m, 4H); ¹³C NMR (100 MHz, D₂O): δ 174.3, 173.8, 161.2, 136.9, 129.8, 49.0, 35.6; HPLC (Waters Atlantis, MeCN/H₂O 15/85, 10 mL/min): 9.4 min I45DC-(Lys)₂ with a yield of 62% AS A white powder: ¹H NMR (400 MHz, D₂O): δ 8.01 (s, 1H), 4.43 (dd, J1=6.0 Hz, J2=3.9 Hz, 2H), 2.86 (t, J=5.4 Hz, 4H), 1.91-1.77 (m, 4H), 1.62-1.56 (m, 4H), 1.42-1.34 (m, 4H) ¹³C NMR (100 MHz, D₂O): δ 174.9, 162.7 (q, TFA), 160.7, 136.5, 129.1, 116.1 (q, TFA), 54.3, 52.9, 39.1, 30.0, 26.2, 21.9; HPLC (Waters Atlantis, MeCN/H₂O 8/92, 10 mL/min): 15 min.

### c) Synthesis 3,5-bis[(Glu)carbonyl]-1H-pyrazole

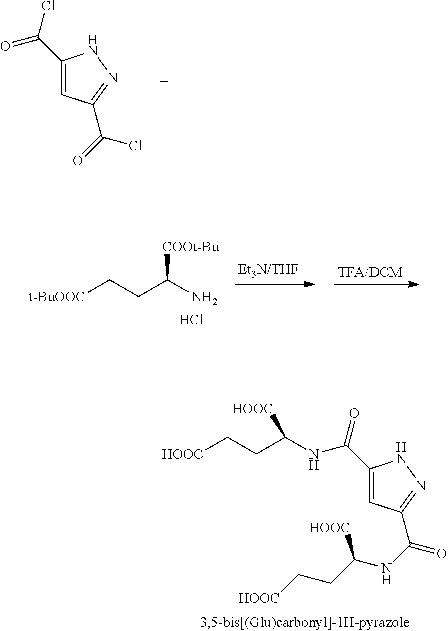

1H-Pyrazole-3,5-dicarbonyl dichloride 384 mg (2 mmol) and THF 10 mL were added to a dry flask. To this suspension at 0° C. was added H-Glu(Ot-Bu)Ot-Bu HCl 1.2 g (4 mmol) and triethyl amine 3 mL (20 mmol). The reaction was stirred overnight at room temperature. After the solvent was removed under vacuum, the tert-butyl protected intermediate was obtained by flash column chromatography. Then, this intermediate was dissolved in 5 mL TFA/DCM (1/1) for 2 hours at room temperature. After all the solvent was removed under vacuum, compound 3,5-bis[(Glu)carbonyl]-1H-pyrazole was purified by HPLC as a white powder 280 mg, yield 34%; HPLC (Waters Atlantis, MeCN/H₂O 15/85, 10 mL/min): 10.5 min ¹H NMR (400 MHz, D₂O): δ 7.09 (s, 1H), 4.45 (dd, J1=6.9 Hz, J2=3.9 Hz, 2H), 2.38 (t, J=5.4 Hz, 4H), 2.18-2.09 (m, 2H), 2.00-1.92 (m, 2H) ¹³C NMR (100 MHz, D₂O): δ 176.9, 174.6, 161.8, 141.6, 106.3, 52.0, 30.0, 25.6.

### d) Synthesis of 4,5-bis[(Glu)carbonyl]-1H-1,2,3-triazole

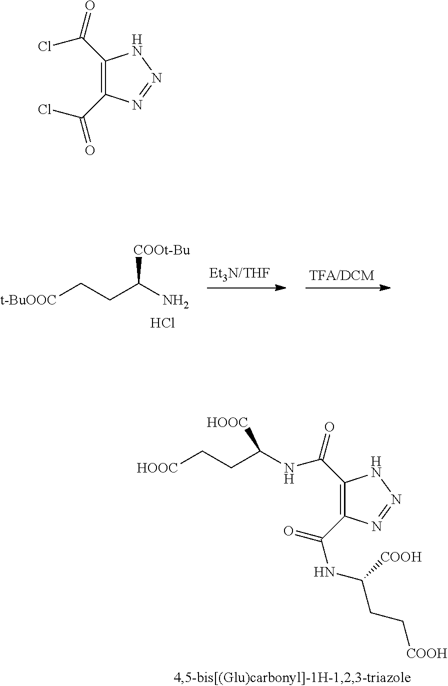

1H-1,2,3-Triazole-4,5-dicarbonyl dichloride 326 mg (1.7 mmol) and THF 10 mL were added to a dry flask. To this suspension at 0° C. was added H-Glu(Ot-Bu)Ot-Bu HCl 1.0 g (3.4 mmol) and triethyl amine 3 mL (20 mmol). The reaction was stirred overnight at room temperature. After the solvent was removed under vacuum, the tert-butyl protected intermediate was obtained by flash column chromatography. Then, this intermediate was dissolved in 5 mL TFA/DCM (1/1) for 2 hours at room temperature. After all the solvent was removed under vacuum, compound 4,5-bis[(Glu)carbonyl]-1H-1,2,3-triazole was purified by HPLC as a light yellow powder 300 mg, yield 43%

¹H NMR (400 MHz, D₂O): δ 4.54 (dd, J1=6.3 Hz, J2=3.6 Hz, 2H), 2.39 (t, J=5.4 Hz, 4H), 2.22-2.14 (m, 2H), 2.05-1.98 (m, 2H) ¹³C NMR (100 MHz, D₂O): δ 176.9, 174.2, 160.4, 137.1, 52.1, 29.8, 25.8; HPLC (Waters Atlantis, MeCN/H₂O 15/85, 10 mL/min): 12.5 min.

### Example 2

### Screening of Azole Heterocycles for CEST Contrast Properties

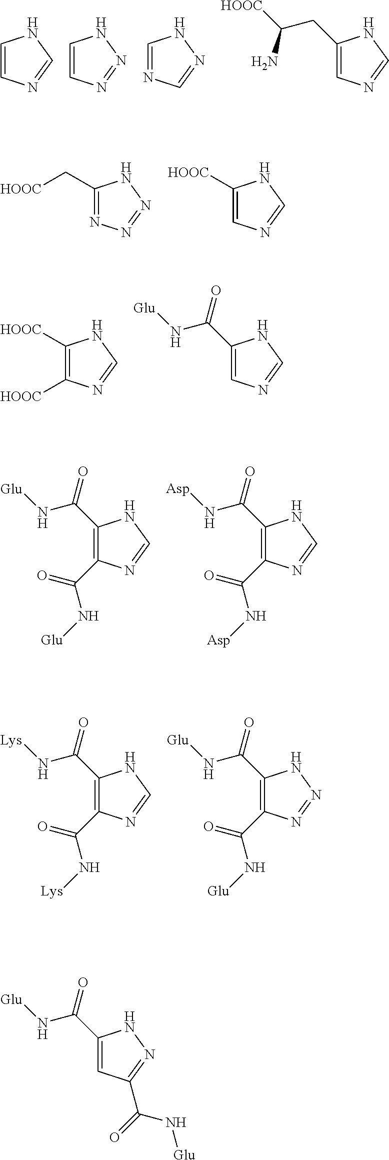

All compounds were dissolved in 0.01M phosphate-buffered saline (PBS) with concentrations of 25 mM and 50 mM. They were then titrated by HCl/NaOH to the pH of 6.2 and 7.4. The solutions were placed into 1 mm capillary tubes and then assembled in a holder for high throughput CEST MR imaging. CEST experiments were taken on a Bruker Biospec 11.7T MR scanner, using a RARE sequence with CW saturation pulse length of 3 seconds and saturation field strength (B1) of 3.6 uT. The CEST Z-spectra were acquired by incrementing the saturation frequency every 0.3 ppm from −12 to 12 ppm for phantoms; TR=6 s, effective TE=17-19 ms, matrix size=96×64. CEST contrast was quantified by MTR_{asym}=(S_{-Δω}−S_{30 Δω})S₀ after a voxel-by-voxel B0 correction, with characterized mean Z-spectra and MTR_{asym} spectra for sample ROIs plotted. I45DCs were found to give the best CEST signal among the azoles tested with 10% or higher contrast observed at 7.5 ppm.

### Example 3

### The Dependence of CEST Contrast on pH and the Determination of the Proton Exchange Rate of I45DCs

I45DCs were dissolved in a 0.01M phosphate-buffered saline (PBS) with several concentrations from 5 mM to 50 mM. They were then titrated using HCl/NaOH to various pH's ranging from 3.5 to 10. The solutions were placed into 1 mm capillary tubes and then assembled in a holder for high throughput CEST MR imaging. CEST experiments were taken on a Bruker Biospec 11.7T MR scanner, using a RARE sequence with CW saturation pulse length of 3 seconds and saturation B1 from 3.6 to 11.4 uT. The CEST Z-spectra were acquired by incrementing saturation frequency every 0.3 ppm from −12 to 12 ppm for phantoms; TR=6 s, effective TE=17-19 ms, matrix size=96×64. CEST contrast was quantified by MTR_{asym}=(S_{-Δω}−S_{+Δω})/S₀ after a voxel-by-voxel B0 correction, with characterized mean Z-spectra and MTR_{asym} spectra for sample ROIs plotted. The CEST contrast of I45DCs showed strong dependence on the pH of the solution.

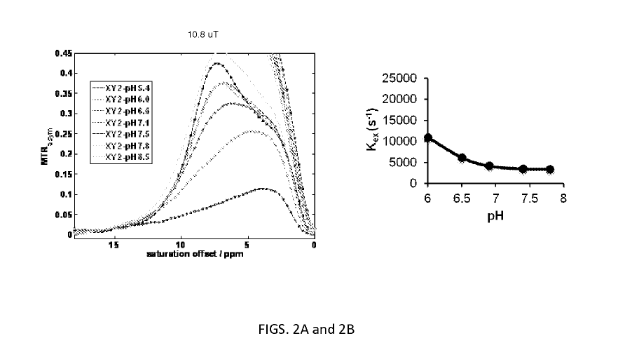

The detected MR-CEST contrast is proportional to saturation efficiency, which is determined by α=(γB₁)²/[(γB₁)²+(k_{sw})²], where B₁ is the saturation field strength, k_{sw} is the exchange rate from solute to water. The k_{sw} was estimated at different pH values. The k_{sw} of I45DCs showed strong dependence on the pH of the solution. A typical result of I45DC-(Glu)₂ is shown in FIGS. 2A-2B).

### Example 4

### Determination of the In Vivo Behavior of I45DCs in the Kidney after Injection

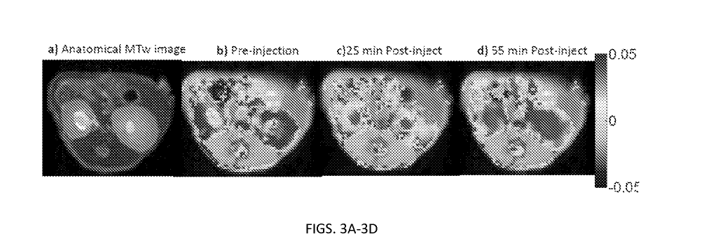

In vivo CEST-MR images were acquired on a Bruker Biospec 11.7T MR scanner. The BALB/c mice weighing 20-25 g (Charles River Laboratories Italia S.r.l., Calco Italia) were maintained under specific pathogen free conditions in the animal facility of Johns Hopkins University. For MRI mice were anesthetized by using 0.5-2% isoflurane and placed in a 23 mm transmit/receive mouse coil. Breath rate was monitored throughout in vivo MRI experiments using a respiratory probe. A 100 μL volume of a 0.25 M I45DC solution in water (pH 7) was slowly injected via a catheter into the tail vein. CEST images of one axial slice were acquired at different time-points pre- and post-injection. The sequence is similar as in phantom study except for TR/TE=5 s/15.12 ms. An example of CEST contrast maps by using I45DC-(Glu)₂ is shown in FIGS. 3 A-3D).

### Example 5

### In Vivo Quantitative pH Mapping Using I45DC-(Glu)₂ as the MRI CEST Agent

### Material and Methods:

Phantom Calibration:

I45DC-(Glu)₂ was dissolved in PBS with conc. of 50 mM, 25 mM, 12 mM and 6.25 mM and pH from 5.4 to 7.5.

In Vivo Preparation:

BALB/c mice weighing 20-25 g (n=3) were anesthetized by isoflurane and placed in a 23 mm transmit/receive mouse coil, with breath rate monitored during MRI. A 100 uL I45DC solution of 0.25 M in water was slowly injected via a catheter into the tail vein.

Imaging:

Images were taken on Bruker 11.7T scanners, using a RARE sequence with CW saturation pulse of B1=5.9 uT, Tₛₐₜ=3 s. For phantoms, saturation frequency incremented every 0.3 ppm from −15 to 15 ppm with TR/TE=6000 ms/17 ms, matrix size=64×48. For in vivo, an axial slice across both kidney carlyx was chosen with thickness of 1.5 mm CEST images with saturation frequencies of [±7.8 ppm, ±7.5 ppm, ±7.2 ppm] and [±4.8 ppm, ±4.5 ppm, ±4.2 ppm] for pH reference were acquired repeatedly every 10 min pre- and post-injection. Image parameters are similar as for phantom except for TR/TE=5 s/15 ms, and CEST contrast was quantified by

MTR_{asym}=(S_{-Δω}−S_{+Δω})/S_{-Δω}.

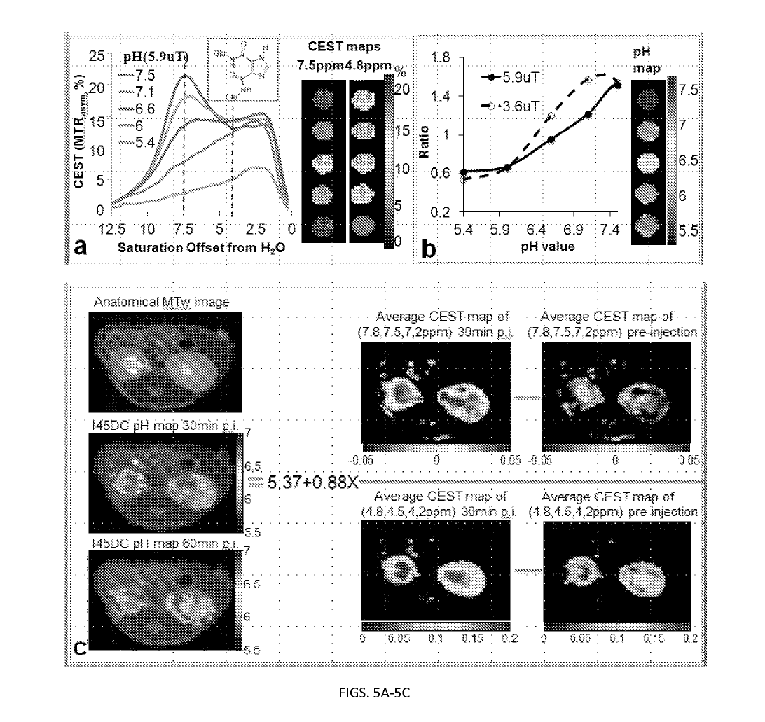

CEST MTR_{asym} spectra of I45DC-(Glu)₂ (FIG. 5A) show 2 broad peaks centered at 7.5 ppm and 2.4 ppm, with the 7.5 ppm peak increasing as pH increases, while the 2.5 ppm-5 ppm part remains relatively constant. As shown in FIG. 5B, the ratio of the 7.5 ppm contrast to contrast at 4.8 ppm is dependent on pH, and can allow neglecting probe concentration. The linearity of the ratio using saturation B1=5.9 uT is better than B1=3.6 uT partially due to a relative fast exchange rate of the heterocyclic NH (kₑₓ=˜4000).

Based on the above results, in vivo studies were performed, which tested how well the pH of the kidney for mice could be estimated, using a 6-offset collection scheme. A calibration function from PBS solutions using the same image conditions was applied for calculating pH, with pH=5.37+0.88*ratio, where ratio=[MTR_{asym} (7.8 ppm)+MTR_{asym} (7.5 ppm)+MTR_{asym} (7.2 ppm)]/[MTR_{asym} (4.8 ppm)+MTR_{asym} (4.5 ppm)+MTR_{asym} (4.2 ppm)]. The baseline of pre-injection contrast was subtracted using either the contrast map or a simple average value for improving CNR. The average kidney pH value is ˜6.3, similar to that disclosed in Longo et al. (Magn Reson Med 2011; 65(1):202-211) (FIG. 5C).

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

### LIST OF REFERENCES

Ward, K. M., Aletras, A. H. & Balaban, R. S. A new class of contrast agents for MRI based on proton chemical exchange dependent saturation transfer (CEST). J. Magn Reson 143, 79-87 (2000).
Goffeney, N., Bulte, J. W., Duyn, J., Bryant, L. H., Jr. & van Zijl, P. C. Sensitive NMR detection of cationic-polymer-based gene delivery systems using saturation transfer via proton exchange. J Am Chem Soc 123, 8628-8629 (2001).
Zhang, S., Merritt, M., Woessner, D. E., Lenkinski, R. E. & Sherry, A. D. PARACEST agents: modulating MRI contrast via water proton exchange. Acc Chem Res 36, 783-790 (2003),
Bar-Shir, A. et al., J, Am Chem. Soc. 2013; Ratnakar, S. J. et al., J. Am Chem, Soc, 2012, 134, 5798.
Liu, a et al., Magn Reson Med. 2012, 67, 1106; Longo, D. L. et al., Magn Reson Med. 2012, doi: 10.1002/mrm. 24513; Li., Y. et al. Contrast Media Mol imaging 2011, 6, 219.
Aime, S. et al, Angew Chem Int Ed Engl 2005, 44, 1813,
Chan, K. W. et al., Nat Mat 2013, 12, 268; Liu, G. et al., NMR in Biomedicine 2013, doi: 10.1002/nbm.2899.
van Zijl P C, Yadav N N. Chemical exchange saturation transfer (CEST): what is in a name and what isn't? Magn Reson Med 2011; 65(4):927-948.
G. Liu, X. Song, K. W. Y. Chan, M. T. McMahon, “Nuts and Bolts of CEST Imaging”, NMR in Biomed. 2013, Doi: 10.1.002/nbm.2899
Sherry A D, Woods M. Chemical exchange saturation transfer contrast agents for magnetic resonance imaging. Annual Review of Biomedical Engineering 2008; 10:391-411.
G. Liu, A. A. Gilad, J. W. M. Bulte, P. C. M. van Zijl, M. T. McMahon. High-Throughput Screening of Chemical Exchange Saturation Transfer MR Contrast Agents. Con. Media. & Imag. 2010; 5(3): 162-170,
Longo D L, Dastru W, Digilio U, Keupp J, Langereis S, Lanzardo S, Prestigio S, Steinbach O, Terreno E, Uggeri F, Aime S. Iopamidol as a responsive MRI-chemical exchange saturation transfer contrast agent for pH mapping of kidneys: In vivo studies in mice at 7 T. Magn Rerun Med 2011; 65(1):202-211.
Sheth V R, Liu G, Li Y, Pagel, M D, improved pH measurements with a single PARACEST MRI contrast agent. Con. Media. & Mol. Imag. 2012 7(1): 26-34.

1. A compound of formula (I), or a salt or stereoisomer thereof: 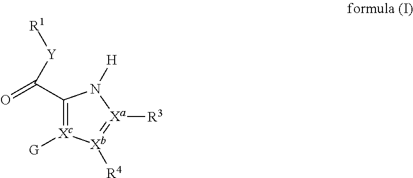
Wherein
X^{a}, X^{b}, and X^{c}, independently, are C, N, O, or S;
Y, on each occurrence, independently is alkyl, NR⁵, O, or S;
G is absent, H, alkyl, cycloalkyl, arylalkyl, cycloalkyl-alkyl, heterocyclic, heteroaryl-alkyl, aryl, heteroaryl, or 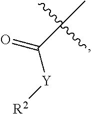
wherein said alkyl, cycloalkyl, arylalkyl, cycloalkyl-alkyl, heterocyclic, heteroaryl-alkyl, aryl, or heteroaryl moiety is optionally substituted;
R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;
R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;
R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or 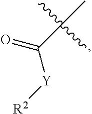
wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and
R⁵ is H, alkyl or —C(O)-alkyl;
provided that said compound is not one of the group of histidine; 4,5-imidazoledicarboxylic acid; 1H-tetrazole-5-acetic acid; and 4-imidazolecarboxylic acid.

2. The compound of claim 1, wherein G is 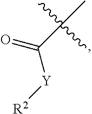
and X^{a} is C.

3. The compound of claim 1, wherein said compound is 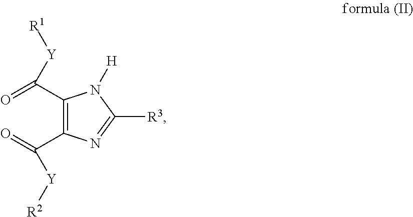
or a salt or stereoisomer thereof;
Wherein
Y, on each occurrence, independently is alkyl, NR⁵, O, or S;
R¹ and R², independently are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O-alkyl moiety is optionally substituted;
R3 is H, halo, alkyl, cycloalkyl, arylalkyl, cycloalkyl-alkyl, heterocyclic, heteroaryl-alkyl, aryl, or heteroaryl, wherein said alkyl, cycloalkyl, arylalkyl, cycloalkyl-alkyl, heterocyclic, heteroaryl-alkyl, aryl, or heteroaryl moiety is optionally substituted; and
R⁵ is H or alkyl.

4. The compound of claim 3, wherein R³ is H.

5. The compound of claim 3, wherein Y is NH.

6. The compound of claim 3, wherein R¹ and R², independently, are (C₁₋₃)alkyl that is optionally substituted by one or more substituents selected from the group of a carboxylic group, an ester group, an amino group, and an amide group.

7. The compound of claim 3, wherein R¹ and R² are the same.

8. The compound of claim 3, wherein said compound is
1) 4,5-bis[(Glu)carbonyl]-1H-imidazole (“I45DC-(Glu)₂”);
2) 4,5-bis[(Lys)carbonyl]-1H-imidazole (“I45DC-(Lys)₂”); and
3) 4,5-bis[(Asp)carbonyl]-1H-imidazole (“I45DC-(Asp)₂”);
or a salt or stereoisomer thereof.

9. The compound of claim 2, wherein X^{c} is C, and X^{a} is N.

10. The compound of claim 9, wherein Y is NH.

11. The compound of claim 9, wherein R¹ and R² are the same and are (C₁₋₃)alkyl that is optionally substituted by one or more substituents selected from the group of a carboxylic group, an ester group, an amino group, and an amide group.

12. The compound of claim 9, wherein said compound is 3,5-bis[(Glu)carbonyl]-1H-pyrazole or 4,5-bis[(Glu)carbonyl]-1H-1,2,3-triazole, or a salt or stereoisomer thereof.

13. The compound of claim 1, wherein G is H, R³ is H, and X^{c} is C.

14. The compound of claim 13, wherein said compound is 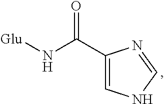
or a salt or stereoisomer thereof.

15. A method selected from the group consisting of:
(I) producing a magnetic resonance (MR) image of a target, said method comprising a step of introducing a magnetic resonance imaging (MRI) contrast agent to said target, wherein said MRI contrast agent is a compound of Formula (A), or a salt or stereoisomer thereof: 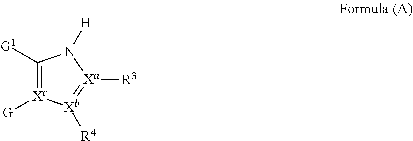
Wherein
X^{a}, X^{b}, and X^{c}, independently, are C, N, O, or S;
G¹ is H, alkyl, or 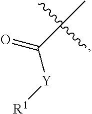
wherein the alkyl is optionally substituted;
G is absent, H, alkyl, cycloalkyl, arylalkyl, cycloalkyl-alkyl, heterocyclic, heteroaryl-alkyl, aryl, heteroaryl, or 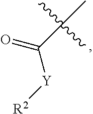
wherein said alkyl, cycloalkyl, arylalkyl, cycloalkyl-alkyl, heterocyclic, heteroaryl-alkyl, aryl, or heteroaryl moiety is optionally substituted;
Y, on each occurrence, independently is alkyl, NR⁵, O, or S;
R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;
R³ is H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;
R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or 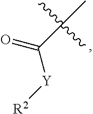
wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and
R⁵ is H, alkyl or —C(O)-alkyl;
(II) diagnosing a tumor in a subject, comprising the steps of
a) introducing to said subject a MRI contrast agent to obtain a conjugation of said MRI contrast agent and a tumor receptor; and
b) detecting or sensing said conjugation,
wherein said MRI contrast agent is a compound of Formula (A), or a salt or stereoisomer thereof: 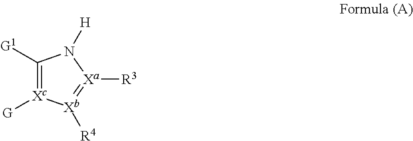
Wherein
X^{a}, X^{b}, and X^{c}, independently, are C, N, O, or S;
G¹ is H, alkyl, or 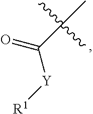
wherein the alkyl is optionally substituted;
G is absent, H, alkyl, cycloalkyl, arylalkyl, cycloalkyl-alkyl, heterocyclic, heteroaryl-alkyl, aryl, heteroaryl, or 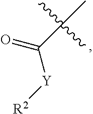
wherein said alkyl, cycloalkyl, arylalkyl, cycloalkyl-alkyl, heterocyclic, heteroaryl-alkyl, aryl, or heteroaryl moiety is optionally substituted;
Y, on each occurrence, independently is alkyl, NR⁵, O, or S;
R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;
R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;
R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or 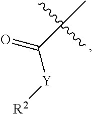
wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and
R⁵ is H, alkyl or —C(O)-alkyl;
(III) detecting a pH value in a biological environment, comprising the steps of
a) introducing to said biological environment a MRI contrast agent; and
b) measuring a chemical shift change of exchangeable protons in said MRI contrast agent;
wherein said MRI contrast agent is a compound of Formula (A), or a salt or stereoisomer thereof: 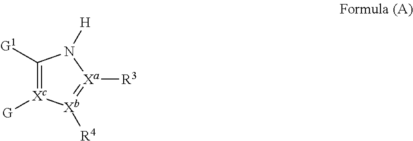
Wherein
X^{a}, X^{b}, and X^{c}, independently, are C, N, O, or S;
G¹ is H, alkyl, or 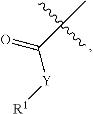
wherein the alkyl is optionally substituted;
G is absent, H, alkyl, cycloalkyl, arylalkyl, cycloalkyl-alkyl, heterocyclic, heteroaryl-alkyl, aryl, heteroaryl, or 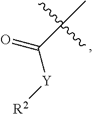
wherein said alkyl, cycloalkyl, arylalkyl, cycloalkyl-alkyl, heterocyclic, heteroaryl-alkyl, aryl, or heteroaryl moiety is optionally substituted;
Y, on each occurrence, independently is alkyl, NR⁵, O, or S;
R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;
R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;
R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or 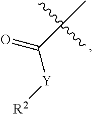
wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and
R⁵ is H, alkyl or —C(O)-alkyl; and
(IV) monitoring delivery of a pharmaceutically active agent in a subject, comprising the steps of
a) administering to said subject said pharmaceutically active agent and a MRI contrast agent; and
b) producing a magnetic resonance (MR) image of said pharmaceutically active agent;
wherein said MRI contrast agent is a compound of Formula (A), or a salt or stereoisomer thereof: 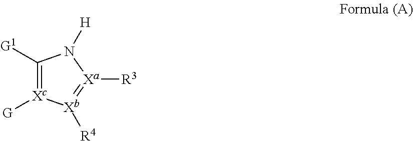
Wherein
X^{a}, X^{b}, and X^{c}, independently, are C, N, O, or S;
G¹ is H, alkyl, or 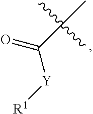
wherein the alkyl is optionally substituted;
G is absent, H, alkyl, cycloalkyl, arylalkyl, cycloalkyl-alkyl, heterocyclic, heteroaryl-alkyl, or 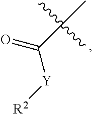
wherein said alkyl, cycloalkyl, arylalkyl, cycloalkyl-alkyl, heterocyclic, heteroaryl-alkyl, aryl, or heteroaryl moiety is optionally substituted;
Y, on each occurrence, independently is alkyl, NR⁵, O, or S;
R¹ and R², independently, are H, alkyl, cycloalkyl, arylalkyl, cycloalkyl-alkyl, heterocyclic, heteroaryl-alkyl, aryl, heteroaryl, —C(O)-alkyl, or —C(O)O-alkyl, wherein said alkyl, cycloalkyl, arylalkyl, cycloalkyl-alkyl, heterocyclic, heteroaryl-alkyl, aryl, heteroaryl, —C(O)-alkyl, or —C(O)O— alkyl moiety is optionally substituted;
R³ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, or —C(O)O-alkyl, wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted;
R⁴ is absent, H, halo, alkyl, alkoxy, cycloalkyl, arylalkyl, cycloalkyl-alkyl, heterocyclic, heteroaryl-alkyl, aryl, heteroaryl, —C(O)-alkyl, —C(O)O-alkyl, or 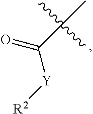
wherein each of said alkyl, alkoxy, cycloalkyl, arylalkyl, cycloalkyl-alkyl, heterocyclic, heteroaryl-alkyl, aryl, heteroaryl, —C(O)-alkyl, and —C(O)O-alkyl moiety is optionally substituted; and
R⁵ is H, alkyl or —C(O)-alkyl.

16. The method of claim 15, wherein said target is a tumor, a biological tissue, a ligand, a therapeutically active agent, or a metal ion.

17. (canceled)

18. The method of claim 15, wherein said method comprising the step of measuring a chemical shift change of exchangeable protons in said MRI contrast agent.

19-20. (canceled)

21. The method of claim 15, wherein said pharmaceutically active agent and said MRI contrast agent are administered concurrently.

22. The method of claim 15, wherein said pharmaceutically active agent and said MRI contrast agent are administered sequentially.

23. The method of claim 15, wherein the method further comprises a step of producing an image through chemical exchange saturation transfer (CEST)-based MRI technique.

24. The method of claim 15, wherein the method further comprises a step of producing an image through frequency labeled exchange (FLEX) imaging technique.

25. The method of claim 15, wherein said method is pH dependent.

26. The method of claim 15, wherein said MRI contrast agent is
1) 4,5-bis[(Glu)carbonyl]-1H-imidazole (“I45DC-(Glu)₂”);
2) 4,5-bis[(Lys)carbonyl]-1H-imidazole (“I45DC-(Lys)₂”);
3) 4,5-bis[(Asp)carbonyl]-1H-imidazole (“I45DC-(Asp)₂”);
4) 3,5-bis[(Glu)carbonyl]-1H-pyrazole;
5) 4,5-bis[(Glu)carbonyl]-1H-1,2,3-triazole;
6) 4,5-imidazoledicarboxylic acid;
7) 1H-tetrazole-5-acetic acid;
8) 4-imidazolecarboxylic acid;
9) imidazole;
10) 1H-1,2,3-triazole;
11) 1H-1,2,4-triazole;
12) 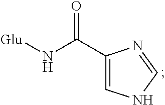
or a salt or stereoisomer thereof.

27. The method of claim 15, wherein said MRI contrast agent is
1) 4,5-bis[(Glu)carbonyl]-1H-imidazole (“I45DC-(Glu)₂”);
2) 4,5-bis[(Lys)carbonyl]-1H-imidazole (“I45DC-(Lys)₂”); or
3) 4,5-bis[(Asp)carbonyl]-1H-imidazole (“I45DC-(Asp)₂”);
or a salt or stereoisomer thereof.

28. A composition selected from the group consisting of:
(I) a kit comprising one or more MRI contrast agents of Formulae (A), and instructions for producing an image thereof; and
(II) a pharmaceutical composition comprising an effective amount of a pharmaceutically active agent, and one or more MRI contrast agents of Formulae (A).

29. (canceled)

30. The pharmaceutical composition of claim 28, wherein said pharmaceutically active agent is a chemotherapeutic drug.
